(19) United States
(12) Patent Application Publication
(10) Pub. No.: US 2024/0331813 A1
(43) Pub. Date: Oct. 3, 2024

(54) COMPUTER-IMPLEMENTED METHOD AND DEVICE FOR CARRYING OUT A MEDICAL LABORATORY VALUE ANALYSIS

(71) Applicant: Robert-Bosch-Krankenhaus GMBH, Stuttgart (DE)

(72) Inventors: Nico Schmid, Stuttgart (DE); Severin Schricker, Stuttgart (DE)

(21) Appl. No.: 18/251,058

(22) PCT Filed: Oct. 25, 2021
(86) PCT No.: PCT/EP2021/079474

§ 371 Date: Apr. 28, 2023

(30) Foreign Application Priority Data
Nov. 9, 2020 (DE) 102020214050.2

(51) Int. Cl. G16H 10/40; G16H 50/20

(52) U.S. Cl. CPC G16H 10/40; G16H 50/20

(57) ABSTRACT

A computer-implemented method for providing at least one predicted value for at least one medical laboratory variable, in particular for use in a medical laboratory value analysis. The method includes: providing at least one laboratory value progression which specifies a progression of historical laboratory values of the at least one laboratory variable at at least two historical points in time; ascertaining at least one laboratory variable feature for each of the at least one laboratory variable from the corresponding laboratory value progression; determining the at least one predicted value at a predetermined prediction time on the basis of a trained, data-based prediction model and on the basis of the at least one laboratory variable feature for each of the at least one laboratory value progression.

### FIELD

The present invention relates to the evaluation of medical laboratory variables, in particular for hematology, urine diagnostics, clinical chemistry, and the like. In particular, the present invention relates to measures for the patient-specific specification of reference ranges for identifying pathological deviations.

### BACKGROUND INFORMATION

Laboratory values for medical diagnostics, in particular for hematology, clinical chemistry, or urine diagnostics, are generally evaluated by medical personnel. These laboratory values are usually obtained by a medical laboratory or similar facilities and are made available to a doctor together with reference ranges specific to these values. These reference values are usually “normal ranges” substantiated by studies, such as the 2.5%-97.5% interquantile range of a healthy population, i.e., the range within which the value in question can be observed for 95 out of 100 healthy people. In some cases, reference values are also adjusted on the basis of gender, age, weight, or other patient features. Laboratory results of which the values are outside this reference range are separately marked to highlight the deviation/pathology thereof.

Furthermore, for particularly critical values, i.e., results that signify a direct risk to the patient, many laboratories implement separate processes to rapidly provide this information to the doctor.

There is no provision for a progression of laboratory variables over time to be taken into account in the evaluation in an automatic, structured, and standardized manner. If any such analysis of the progression is even carried out at all, this is done by the doctor in a manual, often subjective and intuitive, manner.

The very large number of different laboratory variables in modern medicine, the often unclear and frequently unknown interplay therebetween, the lack of expert knowledge, redacted data sources, and the lack of statistical knowledge make it virtually impossible for a doctor to identify a pathological deviation which has not been detected by the above-mentioned standard method. At the same time, this results in a missed opportunity to identify early on problematic changes in a patient's state of health which become noticeable within the standard reference range of a corresponding laboratory value and to initiate corresponding medical measures.

### SUMMARY

According to the present invention, a method for providing a medical laboratory value analysis and a device are provided.

Example embodiments of the present invention are disclosed herein.

According to a first aspect of the present invention, a computer-implemented method for providing at least one predicted value for at least one medical laboratory variable, in particular for use in a medical laboratory value analysis, is provided. According to an example embodiment of the present invention, the method comprises the following steps:
providing at least one laboratory value progression which specifies a progression of historical laboratory values of the at least one laboratory variable at at least two historical points in time;
ascertaining at least one laboratory variable feature for each of the at least one laboratory variable from the corresponding laboratory value progression;
determining the at least one predicted value at a predetermined prediction time on the basis of a trained, data-based prediction model and on the basis of the at least one laboratory variable feature for each of the at least one laboratory value progression.

Furthermore, according to an example embodiment of the present invention, the prediction model can additionally be configured to take into account, in addition to the at least one laboratory variable feature, patient data, such as age, gender, BMI, and other biometric data, such as height, weight, and/or a diagnosis, a finding, a treatment, and/or a medication.

Generally, laboratory variables, such as variables for hematology or urine diagnostics, are usually statically assessed by a doctor on the basis of the laboratory-marked pathological values of the laboratory variables. In general, something is classified as pathological according to specified reference limit values or reference ranges. The progression of laboratory values over time is not assessed in an automated manner. Therefore, problematic trends, in particular in previously unremarkable values, i.e., values of laboratory variables that vary within the medical reference range but still indicate a pathology, can be easily overlooked.

The above computer-implemented method is intended to provide automated evaluation of progressions of medical laboratory variables and to specify, for each variable, an accordingly adjusted reference range that gives an indication of pathological deviations in the laboratory value in question. To do this, at least one laboratory variable feature characterizing the progression of the corresponding laboratory variable is extracted from the respective laboratory value progressions.

A computer-assisted assessment of laboratory value progressions is able to take into account all the available laboratory values and their correlations, and optionally further patient data such as age, gender, weight, height, medical history, pre-existing conditions, and the like, with the aid of an evaluation model, and to provide the doctor with assistance in interpreting a laboratory value by ascertaining the reference range for each laboratory value in a model-based manner and providing it in addition to the laboratory value. This allows medical professionals to assess each laboratory value, not only with regard to a “normal range,” such as the 2.5%-97.5% interquantile range of a healthy population, but also on the basis of an individual reference range and taking into account the historical progressions of laboratory variables.

Furthermore, according to an example embodiment of the present invention, the prediction model can be trained to provide, on the basis of the laboratory variable features, at least one predicted laboratory value which corresponds to the at least one predicted value at the predetermined prediction time.

In particular, a progression of predicted values can be ascertained at a plurality of prediction times to determine a point in time at which the predicted value exceeds a predetermined limit value, wherein, in particular, the point in time can in turn determine a point in time for a medical intervention, such as administering medication.

Alternatively, according to an example embodiment of the present invention, the prediction model can be trained to provide, on the basis of the laboratory variable features, at least one predicted quantile value as the at least one predicted value at the predetermined prediction time. The quantile value can specify an upper or lower limit value of a reference range for the at least one laboratory variable at the prediction time, wherein the result of a comparison of a current laboratory value of the at least one laboratory variable with the corresponding quantile value at the current point in time is indicated as the prediction time.

The laboratory variable features for each of the laboratory variables can include one or more of the following features:
a minimum value of the historical laboratory values, different quantile values, such as the first and the third quartile and the median,
the mean value of the historical laboratory values,
a maximum value of the historical laboratory values,
a standard deviation of the historical laboratory values,
a length of time by which the last-captured historical laboratory value is behind the current point in time,
a length of time by which the penultimate historical laboratory value is behind the current point in time,
a length of time by which the oldest laboratory value is behind the current point in time,
a mean value of the time intervals between the capturing times of the historical laboratory values,
the most recent historical laboratory value,
the second most recent historical laboratory value,
a value of the last gradient between the second most recent and the most recent historical laboratory value,
a length of time until the first outlier of the historical laboratory values,
a point in time for the most recent outlier of the historical laboratory values,
a number of historical laboratory values classified as outliers,
a maximum rise between two successively captured historical laboratory values,
a minimum drop between two successive historical laboratory values,
an estimated linear offset of the historical laboratory values,
an estimated linear increase in the historical laboratory values,
an estimated linear prediction of the historical laboratory values, and
the number of historical laboratory values.

At least one of the at least one laboratory variable feature can be dependent on the predetermined prediction time.

According to further specific embodiments of the present invention, the prediction model can comprise a deep neural network, a convolutional neural network, a recurrent neural network, a support vector machine, a random-forest model, a hidden Markov chain model, or a generalized linear model.

According to a further aspect of the present invention, a method for training a data-based prediction model, in particular for use with the above method, is provided, comprising the following steps:
providing at least one laboratory value progression of at least one laboratory variable, wherein each of the at least one laboratory value progression specifies a progression of historical laboratory values of the at least one laboratory variable at at least three historical points in time;
ascertaining at least one laboratory variable feature for each of the at least one laboratory variable from the corresponding at least one laboratory value progression before a label time;
compiling training data sets by forming each training data set from the at least one laboratory variable feature for the at least one laboratory variable and the laboratory value of the at least one laboratory variable at the label time as the label;
training the data-based prediction model on the basis of the training data sets.

### BRIEF DESCRIPTION OF THE DRAWINGS

Specific embodiments of the present invention will be explained in more detail below on the basis of the figures.

FIG. 1 shows a system for carrying out a laboratory value analysis, according to an example embodiment of the present invention.

FIG. 2 is a flow diagram illustrating a method for carrying out a laboratory value analysis, according to an example embodiment of the present invention.

FIG. 3A to 3C are corresponding graphical representations of progressions and trends for a laboratory variable of an exemplary patient and the predicted values determined by the method, according to an example embodiment of the present invention.

### DETAILED DESCRIPTION OF EXAMPLE EMBODIMENTS

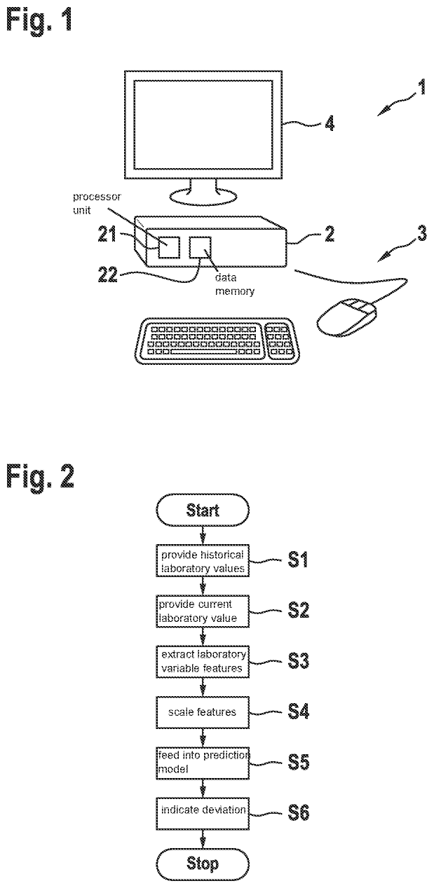

FIG. 1 shows a conventional computer system 1 comprising a computer unit 2, an input apparatus 3, and an output apparatus 4 in the form of a monitor or the like. The computer unit 2 is used to process historical laboratory values for a patient using a processor unit 21 based on software and to provide assistance in the evaluation of current laboratory values. The software and the laboratory values for patients are stored in a data memory 22 in the computer unit 2. Furthermore, the data memory 22 stores parameters of a prediction model as described in the following. The software is executed by the processor unit 21 and accesses the laboratory values stored in the data memory 22. Furthermore, the computer unit 2 can obtain and process laboratory values, input automatically or manually, of laboratory variables.

FIG. 2 is a flow diagram illustrating a method for the laboratory value analysis which is intended to make it easier for medical personnel, such as a doctor, to potentially identify an indication of a possible pathology in current and future laboratory values and to take treatment measures where necessary on the basis of the results of the laboratory value analysis.

In the method, in step S1, historical laboratory values are provided from the data memory of the computer unit. The historical laboratory values can be laboratory values for hematology, urine diagnostics, clinical chemistry, swab diagnostics, or the like. In particular, depending on the type of laboratory variables captured, a multitude of laboratory values can be captured for the laboratory variables. For example, the following laboratory variables can be determined in a hematological blood test: AST/GOT, leukocytes, erythrocytes, hemoglobin, hematocrit, MCV, MCH, MCHC, thrombocytes, pH (AB status), pCO₂ (AB status), standard bicarbonate, O₂ saturation, lactate, ionized Ca, C-reactive protein, glucose, sodium, potassium, potassium from BGA, calcium, creatinine, GFR-MDRD, urea, INR (therapeutic range), PTT, and the like.

In step S2, current laboratory values of the laboratory variables are provided for the current point in time. These are the result of a recently performed test and form the basis for determining the patient's current state of health. The current laboratory values can be manually input into the computer system 1 or automatically received over a communication link. The current laboratory values help the doctor to ascertain the treatment. The following steps are intended to assist the doctor in evaluating the current laboratory values of the laboratory variables taking into account the historical laboratory values and apparent trends therein.

In step S3, laboratory variable features are first extracted from the historical laboratory values. For each of the captured laboratory variables, a number of laboratory variable features are extracted which are dependent on the progression at least in part. These are extracted expressly without incorporating the current laboratory values at the current point in time. For each of the 26 laboratory variables set out above by way of example, a number of laboratory variable features are extracted, for example 23 laboratory variable features. The laboratory variable features for each of the laboratory variables can contain the following features:
a minimum value of the historical laboratory values,
a first quartile value of the historical laboratory values,
a median value of the historical laboratory values,
a mean value of the historical laboratory values,
a third quartile value of the historical laboratory values,
a maximum value of the historical laboratory values,
a standard deviation of the historical laboratory values,
a length of time by which the last-captured historical laboratory value is behind the current point in time,
a length of time by which the penultimate historical laboratory value is behind the current point in time,
a length of time by which the oldest laboratory value is behind the current point in time,
a mean value of the time intervals between the capturing times of the historical laboratory values,
the most recent historical laboratory value,
the second most recent historical laboratory value,
a value of the last gradient between the second most recent and the most recent historical laboratory value,
a length of time until the first outlier of the historical laboratory values,
a point in time for the most recent outlier of the historical laboratory values,
a number of historical laboratory values classified as outliers,
a maximum rise between two successively captured historical laboratory values,
a minimum drop between two successive historical laboratory values,
an estimated linear offset of the historical laboratory values,
an estimated linear increase in the historical laboratory values,
an estimated linear prediction of the historical laboratory values, and
the number of historical laboratory values.

Other features can also be defined. Some of the features are dependent on the prediction time for which a predicted value is intended to be determined.

The feature extraction results in a so-called feature matrix, in which each column represents a laboratory variable feature.

Examples of laboratory variable features are: average time interval between measurements of creatinine, total number of extreme fluctuations observed in the hemoglobin progression, maximum value for sodium, etc. The rows in the feature matrix represent a patient's state at a given point in time (as a sum of their features).

Some machine-learning models (for example, neural networks) make it possible to use raw data. In this case, feature extraction can be dispensed with. The option of directly introducing expert medical knowledge is a reason for using feature extraction, however. For instance, the 23 above-described features are selected together, since they can be considered to be relevant features in laboratory value progressions. Furthermore, laboratory variable features generated in this way provide the option of interpreting later results more easily or directly generating new queries (see feature selection).

In step S4, the individual features, which can vary significantly both in terms of their order of magnitude and their control, are scaled by standardization. For example, scaling to the unit interval can be performed. Alternatively, scaling to a standard normal distribution can also be performed. In principle, standardization before the feature extraction step is also possible. This could be in addition to this standardization step or instead of it.

In a subsequent step S5, the standardized features are fed into a prediction model. The prediction model ascertains a predicted value resulting from the time series of the laboratory variables. To do this, the prediction model is trained to compile a predicted value on the basis of the laboratory variable features.

Since the current point in time is regarded as the prediction time in some of the above-described laboratory variable features, a prediction at the current point in time is possible. For example, the predicted value can specify an estimated value of the laboratory variable in question at the current point in time. For example, this allows a doctor to identify a deviation from a trend that appears in the historical laboratory values, which can indicate an acute illness, for example.

Alternatively, two separately trained prediction models can be provided which ascertain an upper and lower quantile value, for example a 97.5% quantile and a 2.5% quantile, for the prediction time from the previously determined laboratory variable features. These quantiles can specify a reference range for evaluating or interpreting the laboratory variable in question. The reference range specifies the range in which the current, patient-specific laboratory value of the laboratory variable in question should lie or would be expected to lie at the current point in time.

If a deviation from the current reference range determined in a manner tailored to the patient occurs for one or more laboratory variables, this can accordingly be indicated in step S6 for each of the laboratory variables under consideration, for example by a colored marker, such that the corresponding anomaly is indicated to the doctor.

Alternatively or additionally, the prediction model trained to output the current laboratory value can be queried multiple times in order to output, for future points in time, a progression, a progression corridor of reference ranges, or a trend in one or more laboratory variables. In this case, it should be noted that the laboratory variable features are dependent on the prediction time in part, and therefore need to be taken into account in every query.

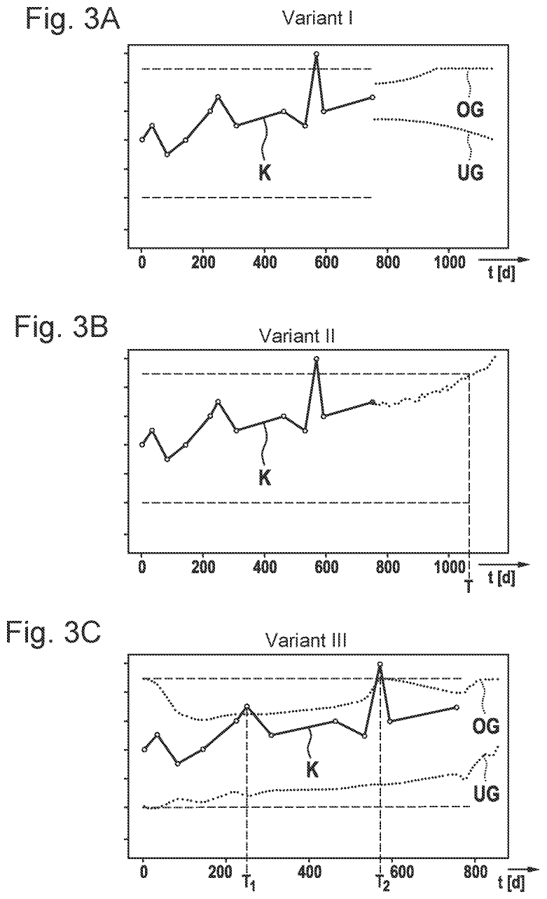

FIG. 3A is a corresponding graphical representation of a trend for an exemplary laboratory variable of an exemplary patient. It shows a personalized prediction for a laboratory variable progression K for the potassium laboratory variable. The progressions OG, UG for the 2.5% and 97.5% quantile, which are shown as dotted lines, have been predicted for the individual patient. The model thus predicts a progression within this range in 95 out of 100 cases. Furthermore, the constant upper and lower limits of the potassium value are shown by dashed lines in accordance with the conventional analysis method.

FIG. 3B shows, as an alternative specific embodiment, a prediction of the mean value into the future and of the anticipated time until this mean value leaves the standard normal range (shown as dashed constant upper and lower limits) at the point in time T in the future.

FIG. 3C shows tailored progressions of upper and lower limits OG, UG as dotted curves for the entire progression of the laboratory variable over time. The points in time T₁, T₂ at which it is identified that said variable leaves the range defined by the upper and lower limits OG, UG can be identified as pathological. A scenario is shown in which just one laboratory value of a laboratory variable is identified as pathological at the point in time T₂ by the standard method. In this scenario, the tailored method set out here allows for earlier identification of a pathological laboratory value at the point in time T₁ in addition to T₂.

The prediction model can additionally be configured to take into account patient data such as age, gender, and other biometric data such as weight, height, and the like, and/or diagnoses, findings, treatments (for example by ICD-10 codes), and/or a medication. In particular, age can also be taken into account in accordance with the prediction time.

The prediction model can be trained on the basis of a multitude of patient data. To do this, time series of laboratory values of laboratory variables can be processed to form training data sets once the time series of the laboratory variables contains three or more points in time. A number of points in time and label data of a label time following the points in time taken into account at which laboratory values have been captured can be taken into account in the time series in order to ascertain the laboratory variable features. In this case, the laboratory variable features can be ascertained at the label time from the points in time of the laboratory values used as label data. This therefore results in training data sets each composed of the laboratory variable features for each of the laboratory variables in question and the label data for each of the laboratory variables as the predicted value to be trained, such as the corresponding laboratory value at the label time, a corresponding lower or upper quantile value at the label time, and patient data where necessary.

Since a high number of laboratory variable features is ascertained, a feature selection step can be performed before the actual method for training the prediction model. A so-called wrapper method can be used for this purpose. This means that the prediction model is applied to different subsets of the entirety of the laboratory variable features of all the laboratory variables, i.e., only to certain combinations of laboratory variable features. Since not every combination can be tested owing to the high number of features, the method follows predetermined heuristics. For example, a forward selection variant can be used in which the laboratory variable features assessed as being the best are successively added to the currently used subset of laboratory variable features to be taken into account. The result is an optimized subset of laboratory variable features for a certain type of predicted value, such as for a predicted value that specifies a 2.5% quantile of a laboratory variable. For example, backward selection, random search, or other so-called Monte Carlo methods, gradient methods, or the like can be used for selecting the predetermined heuristics for selecting the subset. In addition to wrapper methods, other dimensionality reduction methods, such as principal component analysis (PCA), can also be used.

To train the prediction model, the label data are evaluated in accordance with the desired output value. For instance, the label data are predetermined in accordance with the predicted value, which can correspond to the lower quantile value, the upper quantile value, or an estimated value of the corresponding laboratory variable, for example.

Once the subset of the laboratory variable features has been determined, the training data sets determined therefrom and containing the associated predicted values are specified. Neural networks, convolutional neural networks, support vector machines, random-forest models, hidden Markov chain models, generalized linear models, and the like can be used as possible prediction models. Preferably, a support vector machine (SVM) implementation is used, for example having the training parameters of core RGF, gamma grid 0.001 to 10, lambda grid 0.001 to 10, hyperparameter selection, fivefold cross validation, a pinball loss function with weightings and 0.025 and 0.0975 for the lower and upper quantile value. The loss function for training the prediction model can reproduce the query posed to the method.

In the above case, two optimization methods are accordingly performed for each weighting of the loss function of 0.025 and 0.975. The result is two prediction models which predict the 2.5% quantile and the 97.5% quantile, respectively.

To test the prediction model, the training can be applied to 80% of the available data sets. The remaining 20% are used as test data for the final assessment of the model prediction quality. The assessment criteria are dependent on the model variant used in respect of laboratory value model objectives and the like. Since the model is trained independently of the test data, the level of quality obtained by this method is more robust in respect of problems of overfitting, such as assessment by so-called cross validation, and is preferable to the latter.

1-13. (canceled)

14. A computer-implemented method for providing at least one predicted value for at least one medical laboratory variable for use in a medical laboratory value analysis, comprising the following steps:
providing at least one laboratory value progression which specifies a progression of historical laboratory values of the at least one laboratory variable at at least two historical points in time;
ascertaining at least one laboratory variable feature for each of the at least one laboratory variable from the corresponding laboratory value progression;
determining the at least one predicted value at a predetermined prediction time based on a trained, data-based prediction model and based on the at least one laboratory variable feature for each of the at least one laboratory value progression.

15. The method as recited in claim 14, wherein the prediction model is trained to provide, based on the at least one laboratory variable feature, at least one predicted laboratory value at the predetermined prediction time as the at least one predicted value.

16. The method as recited in claim 15, wherein a progression of predicted values is ascertained at a plurality of prediction times to determine a point in time at which the predicted value exceeds a predetermined limit value, wherein the point in time determines a point in time for a medical intervention.

17. The method as recited in claim 15, where in the medical intervention includes administering medication.

18. The method as recited in claim 14, wherein the prediction model is trained to provide, based on the at least one laboratory variable feature, at least one predicted quantile value at the predetermined prediction time as the at least one predicted value, wherein the quantile value specifies an upper or lower limit value of a reference range for the at least one laboratory variable at the prediction time, wherein a result of a comparison of a current laboratory value of the at least one laboratory variable with the corresponding quantile value at the current point in time is indicated as the prediction time.

19. The method as recited in claim 14, wherein the at least one laboratory variable includes at least one variable for hematology, or clinical chemistry, or endocrinology, or blood gas analysis, or autoantibodies, or tumor markers, or urine diagnostics.

20. The method as recited in claim 14, wherein the laboratory variable features for each of the laboratory variables include one or more of the following features:
a minimum value of the historical laboratory values,
different quantile values including a first and a third quartile and a median of the historical laboratory values,
a mean value of the historical laboratory values,
a maximum value of the historical laboratory values,
a standard deviation of the historical laboratory values,
a length of time by which a last-captured historical laboratory value is behind a current point in time,
a length of time by which a penultimate historical laboratory value is behind the current point in time,
a length of time by which an oldest laboratory value is behind the current point in time,
a mean value of time intervals between capturing times of the historical laboratory values,
a most recent historical laboratory value,
a second most recent historical laboratory value,
a value of a last gradient between the second most recent and the most recent historical laboratory value,
a length of time until a first outlier of the historical laboratory values,
a point in time for a most recent outlier of the historical laboratory values,
a number of historical laboratory values classified as outliers,
a maximum rise between two successively captured historical laboratory values,
a minimum drop between two successive historical laboratory values,
an estimated linear offset of the historical laboratory values,
an estimated linear increase in the historical laboratory values,
an estimated linear prediction of the historical laboratory values,
a number of historical laboratory values.

21. The method as recited in claim 14, wherein the prediction model is additionally configured to take into account, in addition to the at least one laboratory variable feature: (i) patient data, including as age, and/or gender, and/or BMI, and and/or other biometric data, including height and/or weight, and/or (ii) a diagnosis, and/or a finding, and/or a treatment, and/or (iii) a medication and/or a medication administration regime.

22. The method as recited in claim 14, wherein at least one of the at least one laboratory variable feature is dependent on the predetermined prediction time.

23. The method as recited in claim 14, wherein the prediction model includes a deep neural network, or a convolutional neural network, or a recurrent neural network, or a support vector machine, or a random-forest model, or a hidden Markov chain model, or a generalized linear model.

24. A method for training a prediction model, comprising the following steps:
providing at least one laboratory value progression of at least one laboratory variable, wherein each of the at least one laboratory value progression specifies a progression of historical laboratory values of the at least one laboratory variable at at least three historical points in time;
ascertaining at least one laboratory variable feature for each of the at least one laboratory variable from the corresponding at least one laboratory value progression before a label time;
compiling training data sets by forming each training data set from the at least one laboratory variable feature for the at least one laboratory variable and the laboratory value of the at least one laboratory variable at the label time as the label;
training the data-based prediction model on the basis of the training data sets.

25. A device configured to provide at least one predicted value for at least one medical laboratory variable for use in a medical laboratory value analysis, the device configured to:
provide at least one laboratory value progression which specifies a progression of historical laboratory values of the at least one laboratory variable at at least two historical points in time;
ascertain at least one laboratory variable feature for each of the at least one laboratory variable from the corresponding laboratory value progression;
determine the at least one predicted value at a predetermined prediction time based on a trained, data-based prediction model and based on the at least one laboratory variable feature for each of the at least one laboratory value progression.

26. A non-transitory machine-readable storage medium on which are stored commands for providing at least one predicted value for at least one medical laboratory variable for use in a medical laboratory value analysis, the commands, when executed by a computer, causing the computer to perform the following steps:
providing at least one laboratory value progression which specifies a progression of historical laboratory values of the at least one laboratory variable at at least two historical points in time;
ascertaining at least one laboratory variable feature for each of the at least one laboratory variable from the corresponding laboratory value progression;
determining the at least one predicted value at a predetermined prediction time based on a trained, data-based prediction model and based on the at least one laboratory variable feature for each of the at least one laboratory value progression.
